(19) United States
(12) Patent Application Publication
(10) Pub. No.: US 2026/0174774 A1
(43) Pub. Date: Jun. 25, 2026

(54) TREATMENTS FOR TOBACCO SMOKE-EXPOSED SUBJECTS INFECTED WITH INFLUENZA VIRUS

(71) Applicant: THE BOARD OF REGENTS OF THE UNIVERSITY OF OKLAHOMA, Norman, OK (US)

(72) Inventors: Jordan P. METCALF, Norman, OK (US); Wenxin WU, Norman, OK (US)

(73) Assignee: THE BOARD OF REGENTS OF THE UNIVERSITY OF OKLAHOMA, Norman, OK (US)

(21) Appl. No.: 19/426,881

(22) Filed: Dec. 19, 2025

Related U.S. Application Data
(60) Provisional application No. 63/737,969, filed on Dec. 23, 2024.

(51) Int. Cl. A61K 31/5383; A61K 31/196; A61K 31/215; A61K 31/351; A61P 31/16; A61P 37/04; C12N 9/99

(52) U.S. Cl. CPC A61K 31/5383; A61K 31/196; A61K 31/215; A61K 31/351; A61P 31/16; A61P 37/04; C12N 9/99

(57) ABSTRACT

Provided herein are methods of treating influenza virus infections in subjects who have been exposed to cigarette smoke. In particular, provided are methods of treating tobacco (e.g., cigarette) smokers or “secondhand smokers” who have an influenza viral infection by administering a CYP1A1 or CYP1B1 inhibitor to the subject.

### CROSS-REFERENCE TO RELATED APPLICATIONS

This application claims the benefit of U.S. Provisional Patent Application Ser. No. 63/737,969, filed Dec. 23, 2024, the content of which is hereby expressly incorporated herein by reference in its entirety.

### GOVERNMENT SUPPORT

This invention was made with government support under Department of Veterans Affairs grant number 101 BX005023 and National Institute of General Medical Sciences grant number 5P20GM103648. The United States government has certain rights in the invention.

### BACKGROUND

Tobacco smoking is a significant public health problem. It is the primary cause of chronic obstructive pulmonary disease (COPD) and predisposes those with COPD to severe respiratory tract infections. CS exposure alone increases the frequency and severity of respiratory tract infections, increases risk of influenza hospitalization, and reduces influenza vaccine effectiveness in the elderly. Human data, using precision-cut lung slices and human bronchial epithelial cells (HBECs), has shown that antiviral responses to influenza A virus (IAV) infection are suppressed by tobacco smoke (TS) exposure. Specifically, RIG-I mRNA, RIG-I protein, and IFN-β mRNA induction by IAV were inhibited by CS. Our results from two mouse models of CS exposure showed that suppression of RIG-I induction and antiviral cytokine responses by CS occurs and is important in an enhanced susceptibility to IAV infection in the lung.

Antiviral drugs are the primary treatment for the flu. If given early in the course of the disease, they can decrease symptoms of illness, shorten the duration of symptoms, and decrease the incidence of serious complications. The CDC recommends four FDA-approved antiviral drugs for treating the flu, including Oseltamivir (Tamiflu), Zanamivir (Relenza), Peramivir (Rapivab), and Baloxavir (Xofluza). These drugs work by preventing the release of viral particles from infected cells and are most effective when taken within 48 hours of the first symptoms. However, recommendations for which drug to use may vary by age, and only oral oseltamivir is recommended for pregnant women. A healthcare professional is best able to determine if antiviral treatment is needed and which drug is most appropriate. There are other, non-pharmaceutical methods to reduce flu symptoms including: antipyretics to decrease fever, gargling salt water, and sitting in a steamy shower, etc.

Oseltamivir does have some shortcomings and disadvantages. For example, common side effects include nausea, vomiting, diarrhea, and headache. These can be particularly troublesome for patients already dealing with flu symptoms. Rarely, Tamiflu has been associated with neuropsychiatric side effects such as confusion, delirium, and abnormal behavior, especially in children. There may be an effect on kidney function. Dosage adjustments may be necessary for individuals with moderate-to-severe kidney disease.

In general, current antiviral therapies have other limitations. For example, for current antivirals to be effective, they must be taken within 48 hours of the onset of flu symptoms. This narrow window can make it less useful if the flu is not diagnosed quickly. Some strains of the influenza virus have already developed resistance to current antivirals, reducing their effectiveness. Also, although rare, serious allergic reactions and skin reactions have been reported. Further, antivirals do not replace the need for an annual flu vaccine. Vaccination remains the primary method for preventing influenza.

However, because vaccination is not 100% effective against the influenza viruses, and current treatments are not always effective, and may have side effects, it is desirable to have additional therapies for treating persons who are afflicted with and influenza infection, particularly those subjects who may be debilitated or immunocompromised. It is to this goal that the present disclosure is directed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows that CYP1B1 is induced by IAV and CSE in A549 cells. A549 cells were incubated with 0% (Mock) and 5% CSE for 24 h. For IAV infection, A549 cells were exposed to IAV PR8 at an MOI of 0.1 for 24 h. (A) CYP1B1 mRNA levels in A549 were assessed by qRT-PCR and normalized to β-actin. Data are expressed as means±SEM. * denotes a significant difference compared to the mock group (P<0.05). (B) CYP1B1 protein expression in A549 was determined by western blot.

FIG. 2 shows that CYP1B1 knockout (CYP KO) increased survival rates of both NS and CS-exposed mice in lethal IAV infection. The mice were intranasally inoculated with IAV at 2000 (A and B) or 1000 (C and D) PFU/mouse. Mortality and body weight were monitored daily. Body weight data were normalized to each mouse's starting body weight. Data are expressed as mean±standard deviation (n≥9 for all groups). Mortality (A) and body weight (B) during lethal IAV infection in NS mice. Mortality (D) and body weight (E) during lethal IAV infection in CS mice. # denotes significant survival rate difference between the CYP1B1 KO CS and WT CS groups, p<0.05. * denotes significant weight loss difference between the CYP1B1 KO CS and WT CS groups, p<0.05.

FIG. 3 shows that lung injury and BALF cellularity. Each mouse was infected intranasally with 500 PFU of IAV. Mock-treated mice were inoculated with PBS. Bronchoalveolar lavage fluid (BALF) or lung tissue was harvested at day 5 after infection. Total immune cells (A), total protein concentration (C) in BALF and ratio of lung/body weight (B) were determined. Data are expressed as means±SEM (n≥4/group). * denotes significant difference between the two groups, p<0.05. ND=no significant difference between the two groups.

FIG. 4 shows the effects of CYP KO on pulmonary histopathology during IAV infection in NS and CS-exposed mice. Each mouse was infected intranasally with 500 PFU of IAV. Mock-treated mice were inoculated with PBS. Animals were sacrificed at 5 days after infection and lung tissue was harvested. Lung tissue sections prepared from the infected mice were fixed, processed and stained with hematoxylin-eosin (A-F). Histopathologic evaluation and scoring of IAV infection were determined by a pathologist in a blind evaluation (G). Compared to the open alveolar spaces in healthy lungs of uninfected of mice with no CS (A, NS Mock), the lungs of mice with IAV infection (B, C, E, F) contained bronchiolar inflammatory infiltrates (blue arrows) that frequently spilled over into the adjacent alveolar spaces (arrowheads). The interstitial space surrounding small and large caliber vessels was also expanded by perivascular edema and lymphocytic infiltrates. Lesions in the lungs of IAV-infected mice featured distinct evidence of diffuse alveolar damage (DAD) characterized by denuded bronchial epithelium (asterisks), alveolar inflammatory infiltrates (arrowheads), and marked intra-alveolar fibrin (arrows). The lungs of healthy, uninfected mice subjected to CS (D, CS Mock) were also histologically normal, with open bronchi/alveoli and minimal alveolar edema. Scale bar=50 μM (20×). The image shown is representative of 5 mouse lungs from each group.

FIG. 5 shows the innate immune responses to influenza infection in mouse lung. Each mouse was infected intranasally with 500 PFU of IAV. Mock-treated mice were inoculated with PBS. Mice were infected with 500 PFU of IAV. The mice were sacrificed at day 5 p.i. and (A) RIG-I, TLR3 and IAV M1 protein mRNA levels (B) cytokines IFN-β, IFN-2 and IL-6 mRNA levels in the lungs were assessed by qRT-PCR and normalized by β-actin levels. Data are expressed as mean±SEM (n=5). * denotes significant difference between the two groups, p<0.05. ND=no significant difference between the two groups.

FIG. 6 shows that IAV-infected CS-exposed CYP KO mice had increased IFN-γ cytokine levels in BALF. Each mouse was infected intranasally with 500 PFU of IAV. Mock-treated mice were inoculated with PBS. BALF were harvested at day 5 post infection. Mock treated mice were inoculated with PBS. Cytokine protein levels were determined by multiplex immunoassay. Data are expressed as mean±SEM (n≥4 per group). * denotes significant difference between the two groups, p<0.05. ND=no significant difference between the two groups.

FIG. 7 shows that IFN-γ and IL-4 were dominantly induced by IAV infection in CYP KO mouse lung. Each mouse was infected intranasally with 500 PFU of IAV. Mock-treated mice were inoculated with PBS. The mice were sacrificed at day 2, 3 and 5 p.i. and IFN-γ and IL-4 mRNA levels in the lungs were assessed by qRT-PCR and normalized by β-actin levels. Data are expressed as mean±SEM (n=5). * denotes significant difference between the two groups, p<0.05. ND=no significant difference between the two groups.

FIG. 8 shows results of flow cytometry analysis of IFN-γ producing cells in mouse lung. Each mouse was infected intranasally with 500 PFU of IAV. Lung cells were isolated at day 5 post infection. (A) lymphoid cell numbers (B) myeloid cell numbers (C) IFN-γ MFI in myeloid cells. Fluorescence Minus One (FMO) were set as negative controls. The graphs represent mean±standard error (n=5). Statistical analysis was performed by unpaired two-tailed t-test. * denotes significant difference between the two groups, p<0.05. ** denotes significant difference between the two groups, p<0.01. *** denotes significant difference between the two groups, p<0.001. ND=no significant difference between the two groups.

FIG. 9 shows that neutralization of IFN-γ abolished enhanced survival in CS-exposed CYP KO mice during lethal IAV infection. (A) IFN-γ administration induced robust RIG-I and IP-10 mRNA induction in mouse lung. C57BL/6 mice were administrated with IFN-γ (5 μg/mouse) intratracheally in a total volume of 50 μl in PBS. After 6 hours, mouse lungs were collected. mRNA levels were assessed by qRT-PCR and normalized to β-actin. Bar graph represents mean±standard deviation (n=3). Mortality (B) and body weight (C) during lethal IAV infection in CS mice. The mice were intranasally inoculated with IAV at 500 PFU/mouse. WT+IFN-γ+PR8 group received IFN-γ (5 μg/mouse) intratracheally at day 3 p.i. CYP KO+ IFN-γ mAb+PR8 received anti-IFN-γ mAb (clone XMG1.2; BioXcell, Lebanon, NH) intraperitoneally (600 μg/mouse) on days 2, 4, 6 p.i. Mortality and body weight were monitored daily. Body weight data were normalized to each mouse's starting body weight. Data are expressed as mean±standard deviation (n>9 for all groups). # denotes significant survival rate difference between the CYP KO+PR8 and CYP KO+IFN-γ mAb+PR8, p<0.05.

FIG. 10 shows that CYP1B1 inhibitor Chlorprothixene increased survival rates of CS-exposed mice in lethal IAV infection. CS mice were exposed to CS for 6 weeks. For CYP1B1 inhibitor treatment groups, the mice were treated with 5 mg/kg of Ticagrelor, 1.3 mg/kg of Chlorprothixene or 1.5 mg/kg of TMS by oral gavage for 9 days starting from the IAV infection day. Placebo group mice were given the solvent (5% DMSO+40% PEG300+5% Tween 80+50% ddH2O). The mice were intranasally inoculated with IAV at 500 PFU/mouse. Mortality and body weight were monitored daily. Body weight data were normalized to each mouse's starting body weight. Data are expressed as mean±standard deviation (n=15 for all groups). Mortality (A) and body weight (B) during lethal IAV infection. (A) * denotes significant survival rate difference between the IAV NS and placebo CS groups by Kaplan-Meier analysis, p<0.05. # denotes no significant survival rate difference between the IAV NS and Chlorprothixene CS groups. (B) * denotes significant weight loss difference between placebo CS and Chlorprothixene CS groups by Tukey's multiple comparisons test, p<0.05.

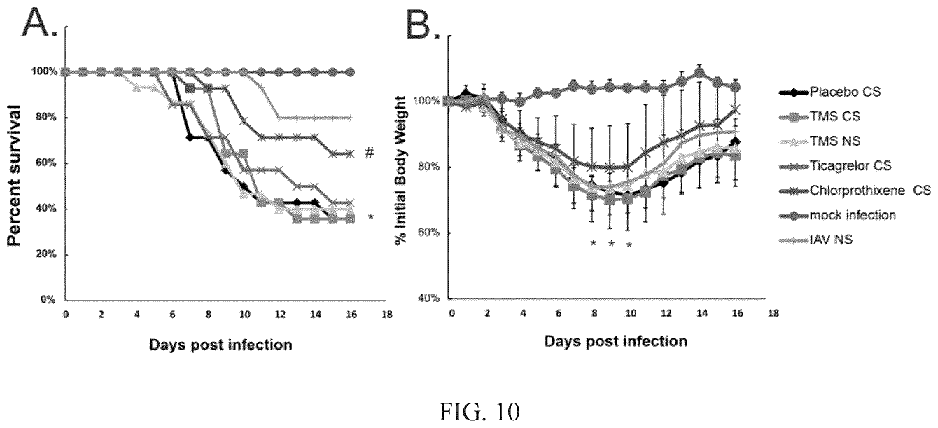

### DETAILED DESCRIPTION

The present disclosure is directed to methods of treating influenza virus infections in subjects who have been exposed to cigarette smoke. In particular, the disclosure is directed to methods of treating tobacco (e.g., cigarette) smokers or “secondhand smokers” who have an influenza viral infection by administering a CYP1B1 inhibitor to the subject.

Examples of CYP1B1 inhibitors that can be used in the methods of the present invention include but are not limited to stilbenes, such as Resveratrol and 2,4,3′,5′-tetramethoxy-trans-stilbene (TMS), flavonoids such as myricetin, apigenin, kaempferol, quercetin, amentoflavone, quercitrin and rutin, and α-naphthoflavone, Flutamide, Pacilitaxel, Mitoxantrone, Docetaxel, Tamoxifen, coumarin, and anthraquinone, chlorprothixene, one-ticagrelor, and Naldifloxacin. Other examples of CYP1B1 inhibitors that can be used include but are not limited to those shown in Tables 2 and 3 below.

As a major site for environmental exposure to inhaled toxicants, the respiratory tract contains many enzymes that metabolize such toxic compounds. A major class of proteins expressed in the bronchial and alveolar epithelia are the cytochrome p450 (CYP) enzymes, of which there are 50 isoforms. Although CYP enzymes ideally metabolize xenobiotics to decrease toxicity, this is not always the case. For example, exposure to dioxin stimulates CYP enzymes that convert arachidonic acid to hydroxyeicosatrienoic acids (HETEs), which play an important role in inflammation. CYP1A1 and CYP1B1 also convert inhaled polycyclic aromatic hydrocarbons to carcinogens. There is also evidence that CYP enzymes, specifically CYP1B1, enhance lung injury during hyperoxic exposure in animal models. CYP1B1−/− mice had ˜50% less lung injury (lung weight/body weight) and lung inflammation than wildtype (WT) mice after 48-72 hours of hyperoxic exposure. In the human bronchial epithelial BEAS-2B cell line, CYP1B1 expression also played a role in hyperoxia induced cytotoxicity. Although it has not been studied in detail, alterations of cytokine expression may play a role in decreased barrier function induced by CYP1B1. Cytokine IL-6 and VEGF mRNA induction by hyperoxia is inhibited in CYP1B1 KO mice, and VEGF contributes to lung injury caused by other stimuli.

Before further describing various embodiments of the present disclosure in more detail by way of exemplary description, examples, and results, it is to be understood that the compounds, compositions, and methods of present disclosure are not limited in application to the details of specific embodiments and examples as set forth in the following description. The description provided herein is intended for purposes of illustration only and is not intended to be construed in a limiting sense. As such, the language used herein is intended to be given the broadest possible scope and meaning, and the embodiments and examples are meant to be exemplary, not exhaustive.

Also, it is to be understood that the phraseology and terminology employed herein is for the purpose of description only and should not be regarded as limiting unless otherwise indicated as so. Moreover, in the following detailed description, numerous specific details are set forth in order to provide a more thorough understanding of the present disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications, and equivalents apparent to those having ordinary skill in the art are included within the scope of the present disclosure. All of the compounds, compositions, and methods and application and uses thereof disclosed herein can be made and executed without undue experimentation in light of the present disclosure. Thus, while the compounds, compositions, and methods of the present disclosure have been described in terms of particular embodiments, it will be apparent to those of skill in the art that variations may be applied to the compounds, compositions, and methods and in the steps or in the sequence of steps of the methods described herein without departing from the concept, spirit, and scope of the inventive concepts.

All patents, applications, published patent applications, and non-patent publications including published articles mentioned in the specification or referenced in any portion of this application are herein expressly incorporated by reference in their entirety to the same extent as if each individual patent or publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular. Where used herein, the specific term “single” is limited to only “one.”

As utilized in accordance with the methods, compounds, and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc. all the way down to the number one (1).

As used in this specification and claims, the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The terms “about” or “approximately,” where used herein when referring to a measurable value such as an amount, a temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a particular embodiment. Further, all references to one or more embodiments or examples are to be construed as non-limiting to the claims.

Use of the word “we,” “us,” and/or “our” as a pronoun in the present disclosure refers generally to laboratory personnel, technicians, or other contributors who assisted in laboratory procedures and data collection and is not intended to represent an inventorship role by said laboratory personnel, technicians, or other contributors in any subject matter disclosed herein.

The term “pharmaceutically acceptable” refers to constructs, compounds and compositions which are suitable for administration to humans and/or animals without undue adverse side effects such as toxicity, irritation and/or allergic response commensurate with a reasonable benefit/risk ratio. The constructs or compounds of the present disclosure may be combined with one or more pharmaceutically-acceptable excipients, including carriers, vehicles, and diluents which may improve solubility, deliverability, dispersion, stability, and/or conformational integrity of the constructs or compounds thereof.

As used herein, “pure” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

The terms “individual,” “subject,” and “patient,” are used interchangeably herein and refer to any warm-blooded subject, particularly mammals, for whom diagnosis, treatment, or therapy is desired, including, but not limited to, dogs, cats, rabbits, rats, mice, guinea pigs, chinchillas, hamsters, ferrets, horses, pigs, goats, cattle, sheep, zoo animals, camels, llamas, non-human primates, including Old and New World monkeys (e.g., cynomolgus macaques, rhesus monkeys, baboons, chimpanzees, gorillas, bonobos, and orangutans), and humans.

The term “active agent” where used herein is intended to refer to a substance which possesses a biological activity relevant to the present disclosure and particularly refers to therapeutic and diagnostic substances which may be used in methods described in the present disclosure. By “biologically active” is meant the ability to modify the physiological system of a cell, tissue, or organism without reference to how the active agent has its physiological effects. Where used herein, unless otherwise noted, the term “active agent” includes pharmaceutically-acceptable salts, hydrates, solvates, and amorphous solids thereof. The recombinant gene construct(s) of the present disclosure may also be referred to herein by the term active agent(s).

“Treatment” refers to therapeutic treatments. “Prevention” refers to prophylactic or preventative treatment measures or reducing the onset of a condition or disease. The term “treating” refers to administering the composition to a subject for therapeutic purposes and/or for prevention.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

The term “effective amount” refers to an amount of an active agent which is sufficient to exhibit a detectable therapeutic or treatment effect in a subject without excessive adverse side effects (such as substantial toxicity, irritation, and allergic response) commensurate with a reasonable benefit/risk ratio when used in the manner of the present disclosure. The effective amount for a subject will depend upon the subject's type, size, and health, the nature and severity of the condition to be treated, the method of administration, the duration of treatment, the nature of concurrent therapy (if any), the specific formulations employed, and the like. The effective amount for a given situation can be determined by one of ordinary skill in the art using routine experimentation based on the information provided herein.

The terms “ameliorate” or “mitigate” mean that a detectable or measurable improvement occurs in a subject's condition, disease, or symptom thereof. A detectable or measurable improvement includes a subjective or objective decrease, reduction, inhibition, suppression, limit, or control in the occurrence, frequency, severity, progression, or duration of the condition or disease, or an improvement in a symptom or an underlying cause or a consequence of the disease, or a reversal of the disease. A successful treatment outcome can lead to a “therapeutic effect” or “benefit” of ameliorating, decreasing, reducing, inhibiting, suppressing, limiting, controlling, or preventing the occurrence, frequency, severity, progression, or duration of a disease or condition, or consequences of the disease or condition in a subject.

A decrease or reduction in worsening, such as stabilizing the condition or disease, is also a successful treatment outcome. A therapeutic benefit therefore need not be complete ablation or reversal of the disease or condition, or of one, most, or all adverse symptoms, complications, consequences, or underlying causes associated with the disease or condition. Thus, a satisfactory endpoint may be achieved when there is an incremental improvement such as a partial decrease, reduction, inhibition, suppression, limit, control, or prevention in the occurrence, frequency, severity, progression, or duration, or inhibition or reversal of the condition or disease (e.g., stabilizing), over a short or long duration of time (hours, days, weeks, months, etc.). Effectiveness of a method or use, such as a treatment that provides a potential therapeutic benefit or improvement of a condition or disease, can be ascertained by various methods and testing assays.

As used herein, the phrase “biologically active” refers to a substance that has activity in a biological system (e.g., in a cell (e.g., isolated, in culture, in a tissue, in an organism), in a cell culture, in a tissue, in an organism, etc.). For instance, a substance that, when administered to an organism, has a biological effect on that organism, is considered to be biologically active. It will be appreciated by those skilled in the art that often only a portion or fragment of a biologically active substance is required (e.g., is necessary and sufficient) for the activity to be present; in such circumstances, that portion or fragment is considered to be a “biologically active” portion or fragment.

The terms “naturally-occurring” and “wild-type” refer to substances that occur in nature, or synthesized substances that are identical to natural substances.

Where used herein, the term “tobacco smoker” or “cigarette smoker” refers to an individual, subject, or patient who smokes tobacco regularly, such as, but not limited to, at least one cigarette, cigar, or pipe-full of tobacco each day or multiple days per week.

Where used herein, the term “secondhand smoke” refers to smoke that comes from the burning of a tobacco product (e.g., cigarette, cigar, pipe) and smoke that is exhaled by tobacco smokers. The term “secondhand smoker” refers to an individual who regularly or frequently spends time in the presence of secondhand smoke and thus inhales tobacco smoke indirectly and/or involuntarily and/or passively.

For purposes of classifying amino acids substitutions as conservative or nonconservative, amino acids are grouped in one non-limiting embodiment as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same group. Non-conservative substitutions constitute exchanging a member of one of these groups for a member of another.

Tables of conservative amino acid substitutions have been constructed and are known in the art. In other embodiments, examples of interchangeable amino acids include, but are not limited to, the following: arginine and lysine; glutamate and aspartate; serine and threonine; glutamine and asparagine; and valine, leucine, and isoleucine. In other non-limiting embodiments, the following substitutions can be made: Ala (A) by leu, ile, or val; Arg (R) by gln, asn, or lys; Asn (N) by his, asp, lys, arg, or gln; Asp (D) by asn or glu; Cys (C) by ala or ser; Gln (Q) by glu or asn; Glu (E) by gln or asp; Gly (G) by ala; His (H) by asn, gln, lys, or arg; Ile (I) by val, met, ala, phe, or leu; Leu (L) by val, met, ala, phe, or ile; Lys (K) by gln, asn, or arg; Met (M) by phe, ile, or leu; Phe (F) by leu, val, ile, ala, or tyr; Pro (P) by ala; Ser(S) by thr; Thr (T) by ser; Trp (W) by phe or tyr; Tyr (Y) by trp, phe, thr, or ser; and Val (V) by ile, leu, met, phe, or ala.

Other considerations for amino acid substitutions include whether or not the residue is located in the interior of a protein or is solvent- (i.e., externally) exposed. For interior residues, conservative substitutions include for example: Asp and Asn; Ser and Thr; Ser and Ala; Thr and Ala; Ala and Gly; Ile and Val; Val and Leu; Leu and Ile; Leu and Met; Phe and Tyr; and Tyr and Trp. For solvent-exposed residues, conservative substitutions include for example: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; and Phe and Tyr.

Compositions or methods “comprising” one or more recited elements may include other elements not specifically recited. For example, a composition that comprises an inhibitor may contain the inhibitor alone or in combination with other ingredients. The phrase “pharmaceutically acceptable salt” refers to pharmaceutically acceptable organic or inorganic salts of a presently-disclosed inhibitor or agent. Exemplary salts include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as (but not limited to) an acetate ion, a succinate ion, or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterions.

The dosage of an administered inhibitor for humans will vary depending upon factors such as (but not limited to) the patient's age, weight, height, sex, general medical condition, and previous medical history. In certain non-limiting embodiments, the recipient is provided with a dosage of the inhibitor that is in the range of from about 1 mg to about 1000 mg as a single infusion or single or multiple injections, although a lower or higher dosage also may be administered. In certain non-limiting embodiments, the dosage may be in the range of from about 25 mg to about 100 mg of the inhibitor per square meter (m²) of body surface area for a typical adult, although a lower or higher dosage also may be administered. Non-limiting examples of dosages of the inhibitor that may be administered to a human subject further include 1 to 500 mg, 1 to 70 mg, or 1 to 20 mg, although higher or lower doses may be used. Dosages may be repeated as needed, for example (but not by way of limitation), once per week for 4-10 weeks, once per week for 8 weeks, or once per week for 4 weeks. It may also be given less frequently, such as (but not limited to) every other week for several months, or more frequently, such as twice weekly or by continuous infusion.

In at least one embodiment, the present disclosure is directed to a dosing regimen involving administration of the inhibitor such as disclosed elsewhere herein. The dosing regimen may comprise multiple dosing cycles (e.g., wherein the first dosing cycle is a step-up, fractionated dosing cycle). The doses may range from about 0.02 mg to about 2.0 mg (e.g., from about 0.02 to about 1.8 mg, from about 0.02 to about 1.6 mg, from about 0.02 to about 1.4 mg, from about 0.02 to about 1.2 mg, from about 0.05 to about 1.8 mg, from about 0.1 to about 1.8 mg, from about 0.4 to about 1.8 mg, from about 0.6 to about 1.8 mg, from about 0.8 to about 1.8 mg, from about 0.5 to about 1.5 mg, from about 0.8 to about 1.2 mg; e.g., about 1 mg), from about 0.05 mg to about 4.0 mg (e.g., from about 0.05 to about 3.5 mg, from about 0.05 to about 3.0 mg, from about 0.05 to about 2.5 mg, from about 0.05 to about 2.2 mg, from about 0.1 to about 3.5 mg, from about 0.5 to about 3.5 mg, from about 1.0 to about 3.5 mg, from about 1.5 to about 3.5 mg, from about 1.8 to about 3.5 mg, from about 1.0 to about 3.0 mg, from about 1.5 to about 2.5 mg; e.g., about 2 mg).

In some embodiments, the dose may range from 50 mg to 200 mg (e.g., from 50 mg to 175 mg, from 50 mg to 150 mg, from 50 mg to 125 mg, from 50 mg to 100 mg, from 50 mg to 75 mg, from 50 mg to 70 mg, from 52 mg to 100 mg, from 52 mg to 75 mg, from 50 mg to 180 mg, from 55 mg to 150 mg, from 55 mg to 100 mg, from 55 mg to 70 mg, from 55 mg to 65 mg, from 58 mg to 62 mg; e.g., about 60 mg). In some embodiments, the dose may be about 60 mg. In some embodiments, the dose is about 1 mg. In some embodiments, the dose is about 2 mg.

In some embodiments, the dose is from 20 mg to 200 mg (e.g., from 20 mg to 175 mg, from 20 mg to 150 mg, from 20 mg to 100 mg, from 20 mg to 75 mg, from 30 mg to 175 mg, from 40 mg to 175 mg, from 45 mg to 175 mg, from 50 mg to 175 mg, from 30 mg to 150 mg, from 40 mg to 100 mg, from 45 mg to 75 mg, from 50 mg to 70 mg, from 55 mg to 65 mg, from 58 mg to 62 mg; about 20 mg, about 30 mg, about 45 mg, or e.g., about 60 mg). In some embodiments, the dose is from about 12 mg to about 48 mg (e.g., from about 12 mg to about 42 mg, from about 12 mg to about 36 mg, from about 12 mg to about 30 mg, from about 18 mg to about 48 mg, from about 18 mg to about 42 mg, from about 24 mg to about 42 mg, from about 27 mg to about 42 mg, from about 24 mg to about 36 mg, from about 27 mg to about 33 mg, from about 28 mg to about 32 mg; e.g., about 24 mg, about 27 mg, about 30 mg, about 33 mg, or about 36 mg).

In some embodiments, the dosing regimen comprises administration of a loading dose, such as from 20 mg to 200 mg (e.g., from 20 mg to 175 mg, from 20 mg to 150 mg, from 20 mg to 100 mg, from 20 mg to 75 mg, from 30 mg to 175 mg, from 40 mg to 175 mg, from 45 mg to 175 mg, from 50 mg to 175 mg, from 30 mg to 150 mg, from 40 mg to 100 mg, from 45 mg to 75 mg, from 50 mg to 70 mg, from 55 mg to 65 mg, from 58 mg to 62 mg; e.g., about 60 mg). In some embodiments, the dose is from about 12 mg to about 48 mg (e.g., from about 12 mg to about 42 mg, from about 12 mg to about 36 mg, from about 12 mg to about 30 mg, from about 18 mg to about 48 mg, from about 18 mg to about 42 mg, from about 24 mg to about 42 mg, from about 27 mg to about 42 mg, from about 24 mg to about 36 mg, from about 27 mg to about 33 mg, from about 28 mg to about 32 mg; e.g., about 24 mg, about 27 mg, about 30 mg, about 33 mg, or about 36 mg).

In some non-limiting embodiments, the inhibitor is provided in a concentration of about 1 nM, about 5 nM, about 10 nM, about 25 nM, about 50 nM, about 75 nM, about 100 nM, about 150 nM, about 200 nM, about 250 nM, about 300 nM, about 350 nM, about 400 nM, about 500 nM, about 550 nM, about 600 nM, about 700 nM, about 800 nM, about 900 nM, about 1 μM, about 2 μM, about 3 μM, about 4 μM, about 5 μM, about 6 μM, about 7 μM, about 8 μM, about 9 μM, about 10 μM, about 15 μM, about 20 μM, about 25 μM, about 30 μM, about 35 μM, about 40 μM, about 45 μM, about 50 μM, about 60 μM, about 70 μM, about 75 μM, about 80 μM, about 90 μM, about 100 μM, about 125 μM, about 150 μM, about 175 μM, about 200 μM, about 250 μM, about 300 μM, about 350 μM, about 400 μM, about 500 μM, about 600 μM, about 700 μM, about 750 μM, about 800 μM, about 900 μM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 100 mM, about 110 mM, about 120 mM, about 130 mM, about 140 mM, about 150 mM, about 160 mM, about 170 mM, about 180 mM, about 190 mM, about 200 mM, about 250 mM, about 300 mM, about 400 mM, about 500 mM, about 600 mM, about 700 mM, about 800 mM, about 900 mM, about 1000 mM, about 1 M, about 1.1 M, about 1.2 M, about 1.3 M, about 1.4 M, about 1.5 M, about 1.6 M, about 1.7 M, about 1.8 M, about 1.9 M, about 2 M, about 3 M, about 4 M, about 5 M, about 6 M, about 7 M, about 8 M, about 9 M, about 10 M, about 15 M, about 20 M, about 25 M, about 30 M, about 35 M, about 40 M, about 45 M, about 50 M, about 75 M, about 100 M, or any range in between any two of the aforementioned concentrations, including said two concentrations as endpoints of the range, or any number in between any two of the aforementioned concentrations.

When administered orally, the presently disclosed inhibitors may be protected from digestion. This can be accomplished either by complexing the construct with a composition to render it resistant to acidic and enzymatic hydrolysis or by packaging the inhibitor in an appropriately resistant carrier such as (but not limited to) a liposome, e.g., such as shown in U.S. Pat. No. 5,391,377.

For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. For topical transdermal administration, the agents are formulated into ointments, creams, salves, powders, and gels. Transdermal delivery systems can also include (for example but not by way of limitation) patches. The presently disclosed constructs can also be administered in sustained delivery or sustained release mechanisms. For example, biodegradeable microspheres or capsules or other biodegradeable polymer configurations capable of sustained delivery of a peptide can be included herein.

For inhalation, the present compositions can be delivered using any system known in the art, including (but not limited to) dry powder aerosols, liquids delivery systems, air jet nebulizers, propellant systems, and the like. For example (but not by way of limitation), the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include (for example but not by way of limitation) air jet nebulizers.

In one aspect, the pharmaceutical formulations comprising inhibitors are incorporated in lipid monolayers or bilayers, such as (but not limited to) liposomes, such as shown in U.S. Pat. Nos. 6,110,490; 6,096,716; 5,283,185; and 5,279,833. In other aspects, non-limiting embodiments of the disclosure include formulations in which the inhibitors have been attached to the surface of the monolayer or bilayer of the liposomes. Liposomes and liposomal formulations can be prepared according to standard methods and are also well known in the art, such as (but not limited to) those disclosed in U.S. Pat. Nos. 4,235,871; 4,501,728; and 4,837,028.

In one aspect, the inhibitors are prepared with carriers that will protect the inhibitor against rapid elimination from the body, such as (but not limited to) a controlled release formulation, including implants and microencapsulated delivery systems. Biodegradable, biocompatible polymers can be used, such as (but not limited to) ethylene vinyl acetate, polyanhydrides, polyglycolic acid, collagen, polyorthoesters, and polylactic acid. Methods for preparation of such formulations will be apparent to those skilled in the art.

The inhibitors in general may be formulated to obtain compositions that include one or more pharmaceutically suitable excipients, surfactants, polyols, buffers, salts, amino acids, or additional ingredients, or some combination of these. This can be accomplished by known methods to prepare pharmaceutically useful dosages, whereby the active compound is combined in a mixture with one or more pharmaceutically suitable excipients. Sterile phosphate-buffered saline is one non-limiting example of a pharmaceutically suitable excipient.

Non-limiting examples of routes of administration of the inhibitor described herein include parenteral injection, e.g., by subcutaneous, intramuscular, or transdermal delivery. Other forms of parenteral administration include (but are not limited to) intravenous, intraarterial, intralymphatic, intrathecal, intraocular, intracerebral, or intracavitary injection. In parenteral administration, the compositions will be formulated in a unit dosage injectable form such as (but not limited to) a solution, suspension, or emulsion, in association with a pharmaceutically acceptable excipient. Such excipients are inherently nontoxic and nontherapeutic. Non-limiting examples of such excipients include saline, Ringer's solution, dextrose solution, and Hanks' solution. Nonaqueous excipients such as (but not limited to) fixed oils and ethyl oleate may also be used. An alternative non-limiting excipient is 5% dextrose in saline. The excipient may contain minor amounts of additives such as (but not limited to) substances that enhance isotonicity and chemical stability, including buffers and preservatives. The inhibitors can be delivered or administered alone or as pharmaceutical compositions by any means known in the art, such as (but not limited to) systemically, regionally, or locally; by intra-arterial, intrathecal (IT), intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa).

Administration of the inhibitor can be (for example but not by way of limitation) parenteral, intravenous, oral, subcutaneous, intra-arterial, intracranial, intrathecal, intraperitoneal, topical, intranasal, or intramuscular. Administration can also be localized directly into a tumor. Administration into the systemic circulation by intravenous or subcutaneous administration is typical. Intravenous administration can be, for example (but not by way of limitation), by infusion over a period such as (but not limited to) 30-90 min or by a single bolus injection.

Formulated compositions comprising the inhibitor can be used (for example but not by way of limitation) for subcutaneous, intramuscular, or transdermal administration. Compositions can be presented in unit dosage form, e.g., in ampoules or in multi-dose containers, with an added preservative. Compositions can also take such forms as suspensions, solutions, or emulsions in oily or aqueous vehicles, and can contain formulatory agents such as suspending, stabilizing, and/or dispersing agents. The compositions may be administered in solution. The formulation thereof may be in a solution having a suitable pharmaceutically acceptable buffer, such as (but not limited to) phosphate, Tris (hydroxymethyl)aminomethane-HCl, or citrate, and the like. Buffer concentrations should be in the range of 1 to 100 mM. The formulated solution may also contain a salt, such as (but not limited to) sodium chloride or potassium chloride in a concentration of 50 to 150 mM. An effective amount of a stabilizing agent such as (but not limited to) mannitol, trehalose, sorbitol, glycerol, albumin, a globulin, a detergent, a gelatin, a protamine, or a salt of protamine may also be included.

Exemplary, non-limiting ranges for a therapeutically or prophylactically effective amount of an inhibitor of the present disclosure include a range of from about 0.001 mg/kg of the subject's body weight to about 100 mg/kg of the subject's body weight, such as but not limited to a range of from about 0.01 mg/kg to about 50 mg/kg, a range of from about 0.1 mg/kg to about 50 mg/kg, a range of from about 0.1 mg/kg to about 40 mg/kg, a range of from about 1 mg/kg to about 30 mg/kg, a range of from about 1 mg/kg to about 20 mg/kg, a range of from about 2 mg/kg to about 30 mg/kg, a range of from about 2 mg/kg to about 20 mg/kg, a range of from about 2 mg/kg to about 15 mg/kg, a range of from about 2 mg/kg to about 12 mg/kg, a range of from about 2 mg/kg to about 10 mg/kg, a range of from about 3 mg/kg to about 30 mg/kg, a range of from about 3 mg/kg to about 20 mg/kg, a range of from about 3 mg/kg to about 15 mg/kg, a range of from about 3 mg/kg to about 12 mg/kg, or a range of from about 3 mg/kg to about 10 mg/kg, or a range of from about 10 mg to about 1500 mg as a fixed dosage.

In some non-limiting methods, the inhibitor is administered the patient every one, two, three, or four weeks, for example. The dosage depends on the frequency of administration, condition of the patient, response to prior treatment (if any), whether the treatment is prophylactic or therapeutic, and whether the disorder is acute or chronic, among other factors.

The frequency of administration depends on the half-life of the inhibitor in the circulation, the condition of the patient, and the route of administration, among other factors. The frequency can be daily, weekly, monthly, quarterly, or at irregular intervals in response to changes in the patient's condition or progression of the disorder treated. An exemplary (but non-limiting) frequency for intravenous administration is between twice a week and quarterly over a continuous course of treatment, although more or less frequent dosing is also possible. Other exemplary (but non-limiting) frequencies for intravenous administration are between once weekly or once monthly over a continuous course of treatment, although more or less frequent dosing is also possible. For subcutaneous administration, an exemplary (but non-limiting) dosing frequency is daily to monthly, although more or less frequent dosing is also possible.

The number of dosages administered may depends on the severity and temporal nature of the disorder (e.g., whether presenting acute or chronic symptoms) and the response of the disorder to the treatment. For acute disorders or acute exacerbations of a chronic disorder, between 1 and 10 doses may be used. Sometimes a single bolus dose, optionally in divided form, is sufficient for an acute disorder or acute exacerbation of a chronic disorder. Treatment can be repeated for recurrence of an acute disorder or acute exacerbation. For chronic disorders, the active agent may be administered at regular intervals, such as (but not limited to) weekly, fortnightly, monthly, quarterly, every six months for at least 1, 5, or 10 years, or for the life of the patient

In certain non-limiting embodiments, the percent identity of two amino acid sequences (or two nucleic acid sequences) can be determined, for example, by aligning the sequences for optimal comparison purposes (e.g., gaps can be introduced in the sequence of a first sequence). The amino acids or nucleotides at corresponding positions are then compared, and the percent identity between the two sequences is a function of the number of identical positions shared by the sequences (i.e., % identity=(no. of identical positions÷total no. of positions)×100). The actual comparison of the two sequences can be accomplished by well-known methods, for example, using a mathematical algorithm. A specific, non-limiting example of such a mathematical algorithm is described in Karlin et al. (Proc. Natl. Acad. Sci. USA, 90:5873-5877 (1993)). Such an algorithm is incorporated into the BLASTN and BLASTX programs (version 2.2) as described in Schaffer et al. (Nucleic Acids Res., 29:2994-3005 (2001)). When utilizing BLAST and Gapped BLAST programs, the default parameters of the respective programs (e.g., BLASTN) can be used. In one non-limiting embodiment, the database searched is a non-redundant (NR) database, and parameters for sequence comparison can be set at: no filters; Expect value of 10; Word Size of 3; the Matrix is BLOSUM62; and Gap Costs have an Existence of 11 and an Extension of 1.

Some non-limiting embodiments provided herein include kits. In some non-limiting embodiments, a kit can include any of the inhibitors as disclosed herein. In some non-limiting embodiments, the inhibitor is lyophilized. In some non-limiting embodiments, the inhibitor is in aqueous solution, or other carrier as described herein. In some non-limiting embodiments, the kit includes a pharmaceutical carrier for administration of the inhibitor. In some non-limiting embodiments, the kit also includes another antiinfluenza virus therapy. Certain non-limiting embodiments of the present disclosure include kits containing components suitable for treatments or diagnosis. A device capable of delivering the kit components by injection, for example, a syringe for subcutaneous injection, may be included in some non-limiting embodiments. Where transdermal administration is used, a delivery device such as hollow microneedle delivery device may be included in the kit in some non-limiting embodiments. Exemplary transdermal delivery devices are known in the art, such as (but not limited to) a hollow Microstructured Transdermal System (e.g., 3M Corp.), and any such known device may be used. The kit components may be packaged together or separated into two or more containers. In some non-limiting embodiments, the containers may be vials that contain sterile, lyophilized formulations of a composition that are suitable for reconstitution. A kit may also contain one or more buffers suitable for reconstitution and/or dilution of other reagents. Alternatively, the construct may be delivered and stored as a liquid formulation. Other containers that may be used include, but are not limited to, a pouch, tray, box, tube, or the like. Kit components may be packaged and maintained sterilely within the containers. Another component that can be included is instructions for the use of the kit for treatment of certain diseases or conditions or for the diagnosis of such.

The kit or article of manufacture may comprise a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be a vial having a stopper pierceable by a hypodermic injection needle). At least one active agent in the composition is an inhibitor as described herein. The label or package insert indicates that the composition is used for treating the condition of choice and further includes dosing information, for example one of the dosing regimens described herein. Moreover, the kit or article of manufacture may comprise (a) a first container with a composition contained therein, wherein the composition comprises an inhibitor as described herein. The kit may further include other materials desirable from a commercial and user standpoint, including other buffers, diluents, filters, needles, and syringes. The term “package insert” is used to refer to instructions customarily included in commercial packages of therapeutic products, that contain information about the indications, usage, dosage, administration, combination therapy, contraindications and/or warnings concerning the use of such therapeutic products.

### Examples

### Methods

### Isolation of Primary Human Bronchial Epithelial Cells

Human bronchial epithelial cells (HBEC) were obtained by bronchoscopy and bronchial brushing with twritten, informed consent from both smoking and non-smoking, healthy, adult volunteers in accordance with a protocol approved by the Institutional Review Board of the University of Oklahoma Health Sciences Center (IRB #2197). The smokers had a smoking history of at least 10 pack years with ½ to 1 pack of cigarettes per day. All smoking and nonsmoking participants were gender, age and ethnicity matched. Three or four separate bronchi were brushed and the cells were rinsed from the brush into 10 ml sterile saline until 5×10⁶ to 1×10⁷ cells total were collected as determined by hemocytometer counts for total and viable cells using trypan blue exclusion. The HBECs were centrifuged at 400×g for 5 min. Cells were resuspended to 5×10⁵ cells/ml in complete Bronchial Epithelial Cell Growth Medium (BEGM; Lonza Group Ltd.); were seeded into collagen coated tissue culture plates (Bio-Coat, BD Biosciences) at a density of 1×10⁵ cells/cm² and were propagated in an incubator at 37° C. in 5% CO₂. After 24 h the cells were washed with HBSS to remove non-adherent cells and fresh complete BEGM was added. When the cultures were near confluence (7-10 days), cells were harvested for RNA extraction.

### Influenza A Virus and Mouse Infection

The IAV strain used in this study was A/PR/34/8 (PR8). The stocks were propagated in Madin-Darby canine kidney (MDCK, ATCC and Manassas, VA) cells following standard procedures. The virus was titered by plaque assay in MDCK cells, aliquots were made and stored at −80° C. LD₅₀ for each viral preparation.

Mice were held in a vertical position while sedated and infected by intranasal instillation of PR8 virus diluted in PBS (50 μl solution). An equal volume of PBS without virus instilled intranasally was used as a control in the mock group. All infected animals were sacrificed by an overdose of isoflurane at 5 days post-infection (p.i.). The animals were meticulously watched both during and after each procedure to make sure they recovered properly. Mice were monitored daily for up to 16 days for clinical symptoms (shivering, inactivity, hunched posture, and piloerection) and their weight was recorded daily, or until the experimental endpoint, whichever came first.

### Whole-Body CS Exposure

C57BL/6 mice were exposed to the smoke of 1R6F reference cigarettes (University of Kentucky, Lexington, KY) for 4 hours per day. Mice receiving CS were gradually brought up to the target exposure over a period of 2 weeks and treated 5 days/week for 6 weeks. Treatment was administered by placing mice in a Plexiglas smoking chamber (Teague Enterprises, Davis, CA). Smoke exposure was standardized to total suspended particles=90 mg/m³, 11% mainstream and 89% sidestream smoke in the chamber of the machine. The exposure level was assessed by measuring serum cotinine, a nicotine metabolite, at 1 h after exposure (Cotinine ELISA Kit, GenWay Biotech Inc.). After six-week exposure, the average serum cotinine was 513+256 ng/ml, near levels in human cigarette smokers. “Nonsmoking” (NS) treatment groups were conducted for the same periods of time, but mice were exposed to filtered room air.

### Bronchoalveolar Lavage (BAL)

Mice were sacrificed using isofluorane. BAL was performed using a closed thorax technique by exposing the trachea, nicking the bottom of the larynx and inserting a ¾-inch 22-gauge cannula into the proximal trachea. The proximal end of the trachea was tied off, and 0.6 ml of sterile PBS was gently introduced into the lungs and recovered. This was repeated 3 times for a total instilled volume of 1.8 ml. Return volume varied by <10% between samples. BAL fluid (BALF) was centrifuged to remove cells and was stored at −20° C. Total protein in BALF was determined by a Pierce BCA Protein Assay Kit (ThermoFisher Scientific, Waltham, MA).

### Multiplex Immunoassay

Cytokine protein levels in the bronchoalveolar lavage fluids (BALF) were determined by multiplex immunoassay (Eve Technologies, Calgary, AB, Canada). All samples were 2-fold diluted in 1% triton X-100 (final) for inactivation of residual IAV.

Measurement of mRNA Expression by Quantitative Real-Time PCR (qRT-PCR)

Total RNA from lung was extracted using a modified TRIzol (Invitrogen, Carlsbad, CA) protocol and spectrophometrically quantitated. The integrity of RNA was verified by formaldehyde agarose gel electrophoresis. Equal amounts (1 μg) of RNA from each sample were reverse-transcripted into cDNA with oligo (dT) SuperScript II First-Strand Synthesis System for RT-PCR (Invitrogen, Carlsbad, CA). Gene specific primers for mouse PRRs, cytokines and the β-actin housekeeping genes were used. The primers' sequences were described in our earlier publication [17]. qRT-PCR was performed using 100 ng sample RNA and SYBR Green (Quanta Biosciences, Gaithersburg, MD) in a Bio-Rad CFX96™ Touch Real-Time PCR Detection System. Results were calculated and graphed from the ΔCT of target gene and normalizer, β-actin.

### Histological and Immunohistochemistry Analysis of Mouse Lung

Mice were euthanized at day 5 after IAV infection, and lungs were fixed in 4% paraformaldehyde in PBS for 24 hours at room temperature before being embedded in paraffin. Paraffin-embedded sections were trimmed to 5 μm according to previously published methods and guidelines [18, 19] and collected onto charged slides before staining with hematoxylin and eosin (H&E) for microscopic evaluation by light microscope. Lung tissues were evaluated for: alveolar damage, hyaline membrane formation, serous exudate/edema, alveolar fibrin deposition, alveolar histiocytes, perivascular infiltrates, type II pneumocyte hyperplasia, peri-bronchial inflammation, smooth muscle hyperplasia, thrombosis, and fibrinoid vasculitis. All tissues were assigned a quantitative histopathological score based on previously described criteria [20, 21]: 0=no apparent pathology/change; 1=minimal change (minimally increased numbers of inflammatory cells); 2=mild change (mild inflammatory infiltrates, damage/necrosis, fibrin deposition and/or exudation); 3=moderate change (as previously described, but more moderately extensive); 4=marked changes (as previously described, but with severe inflammation, damage/necrosis, exudation, vasculitis and/or thrombosis). All tissues were evaluated and scored by a board-certified veterinary pathologist (CAM) blinded to study groups to eliminate bias and ensure scientific rigor.

Isolation of Cells from Lung Tissue

Lungs were perfused with PBS+1 mM EDTA before digestion for 60 min with Liberase (0.1 mg/ml) and DNase I (0.1 mg/ml) (all from Roche) in PBS+0.5% BSA pH 7.4. Lung cells were filtered (70 μM), washed with RPMI+10% FCS or HBSS without Ca²⁺ and Mg²⁺, respectively, and red cells lysed using RBC lysis buffer (BD Biosciences).

### Flow Cytometry

For surface staining, cells were incubated with monoclonal antibodies (mAbs) on ice for 15 min after 5 min of anti-CD16/32 treatment in FACS buffer (PBS, 5% newborn calf serum, 0.1% NaN3). Kits for intranuclear staining (ThermoFisher Scientific) and intracellular cytokine staining (BD Biosciences) were used according to the manufacturer's instructions. Lung cells were identified with a fluorochrome-linked mAb cocktail, including CD45.2-FITC, IFN—Y—PE, CD11c-PECy7, Ly6G-BV786, CD11b-PercpCy5.5, MHCII-AF700, SiglecF-BV421, Ly6C-PEDazzle594, CD24-BV605, and CD64-APC. mAbs were purchased from BD Biosciences, Biolegend, Tonbo Biosciences, and ThermoFisher Scientific. Live/dead cell discrimination was done with a fixable Zombie Aqua™ dye (Biolegend, San Diego, CA). For Mean Fluorescence Intensity (MFI), Fluorescence Minus One (FMO) were set as negative controls, which were the addition of all fluorescently labeled antibodies in the panel minus one to see the influence of each fluorophore on the panel and its spread into neighboring channels. Samples were acquired on an LSRII instrument containing four lasers (BD Biosciences) and analyzed using FlowJo software (Treestar, Ashland, OR).

### Statistical Analysis

Statistical significance was determined by one-way ANOVA with Student-Newman-Keuls post hoc correction for multiple comparisons. Survival rate significance was determined by log rank test. Statistical analysis for flow cytometry data was performed by unpaired two-tailed t-test. For RT-PCR results, the p value was calculated from the ΔCt values from different experimental groups. Significance was considered as p<0.05.

### Results

CYP1B1 is highly expressed in HBECs from cigarette smokers and cigarette smoke extracts (CSE)-treated lung epithelial cells. As CS exposure worsens outcomes during IAV infection in mice and humans, we asked whether CS exposure modulated expression of any genes that could predispose to lung injury during IAV infection. We isolated HBECs from 10 normal nonsmokers and 10 age (±5 years), sex, and ethnicity matched smokers we recruited through an IRB approved protocol. RNA was isolated from freshly isolated cells and whole transcriptomic analysis was performed, and the results were analyzed (Table 1).

Table 1 shows the ten most upregulated genes in human bronchial epithelial cells (HBECs) isolated from smokers. HBECs were isolated from 10 normal nonsmokers and 10 age (±5 years), sex, and ethnicity matched smokers recruited through an IRB approved protocol. RNA was isolated from freshly isolated cells and whole transcriptomic analysis was performed, and the results were analyzed. Of the ten most upregulated genes, CYP1B1 is induced 6-fold (FDR 0.0001) in smokers vs. nonsmokers, and has the second greatest induction of any gene in these subjects.

TABLE 1
Ten Genes Most Upregulated in Smoker vs. Nonsmokers

| | | | FDR |
|---|---|---|---|
| Gene | | Fold- | (adj P |
| Symbol | Gene Name | Change | value) |
| AKR1B10 | aldo-keto reductase family 1, | 8.53 | 1.190E−04 |
| | member B10 (aldose reductase) | | |
| CYP1B1 | cytochrome P450, family 1, | 5.98 | 4.393E−03 |
| | subfamily B, polypeptide 1 | | |
| SLC7A11 | solute carrier family 7 (anionic | 4.79 | 1.639E−03 |
| | amino acid transporter light chain, | | |
| | xc-system), member 11 | | |
| CEACAM5 | carcinoembryonic antigen-related | 3.62 | 2.012E−03 |
| | cell adhesion molecule 5 | | |
| AKR1C2 | aldo-keto reductase family 1, | 3.50 | 5.533E−05 |
| | member C2 | | |
| CYP4F11 | cytochrome P450, family 4, | 3.44 | 6.118E−04 |
| | subfamily F, polypeptide 11 | | |
| CABYR | calcium binding tyrosine-(Y)- | 3.30 | 1.739E−04 |
| | phosphorylation regulated | | |
| ADH7 | alcohol dehydrogenase 7 (class | 3.26 | 1.045E−07 |
| | IV), mu or sigma polypeptide | | |
| MUCL1 | mucin-like 1 | 3.06 | 1.144E−04 |
| ALDH3A1 | aldehyde dehydrogenase 3 family, | 3.01 | 3.982E−07 |
| | member A1 | | |

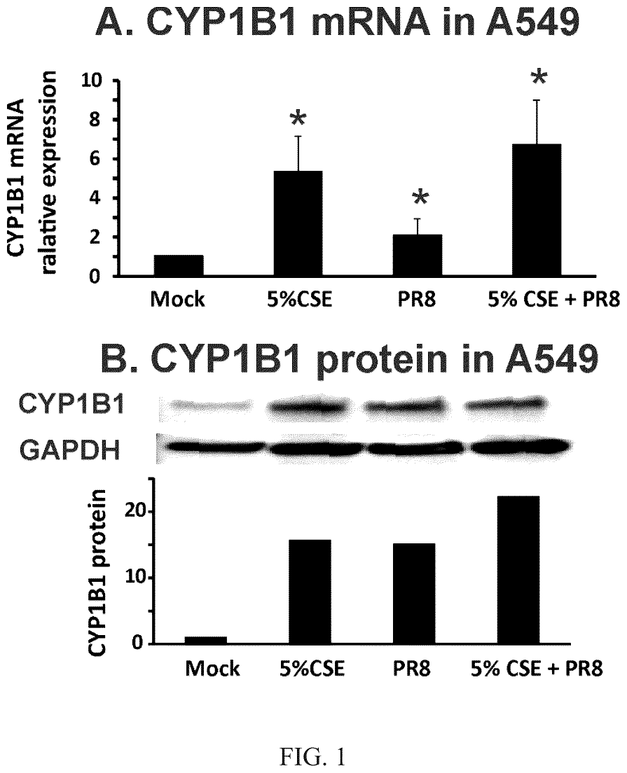

The CYP1B1 gene is induced 6-fold (FDR 0.0001) in smokers vs. nonsmokers, and has the second greatest induction of any gene in these subjects (Table 1). We further confirmed induction of CYP1B1 in the A549 human lung epithelial cell line. A549 cells were treated with CSE 5% for 24 h, then infected with IAV. Both CSE and IAV infection induced CYP1B1 production in A549 cells transcriptionally and translationally (FIG. 1A and B). We investigated whether CYP1B1 plays a role in lung injury in CS exposed mice during IAV infection.

### CYP KO Improves Survival in CS-Exposed Mice During Lethal IAV Infection

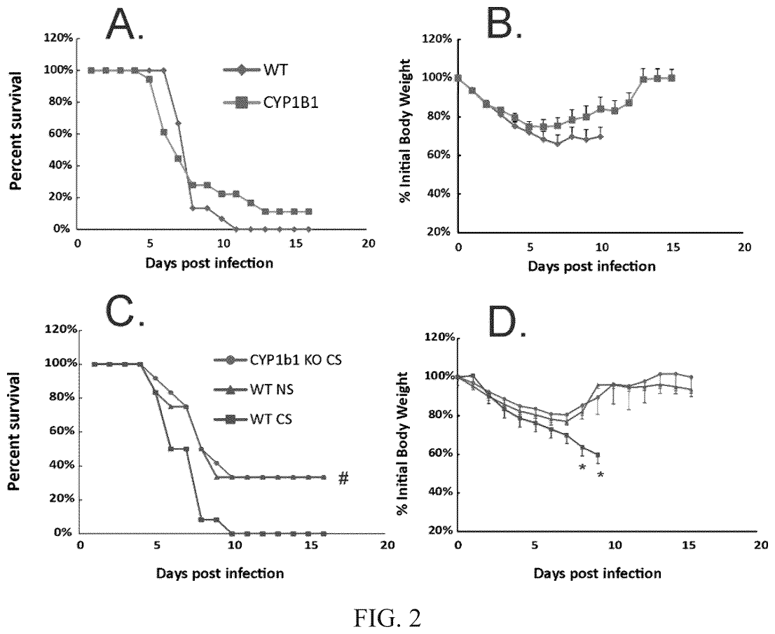

We obtained C57BL/6 CYP1B1-\-mice (gift of Dr. F. Gonzalez) from NIH. First, to test whether CYP KO improves mortality in NS mice, we inoculated animals with a lethal dose of PR8 IAV (2000 PFU). Death was recorded when mice were found dead in the cage or at 70% of original body weight. CYP1B1-\-mice appeared to have decrease mortality a little although the survival curve difference did not reach statistical significance (FIG. 2A). Weight loss occurred in both infected groups (FIG. 2B). Again, the body weight of CYP1B1−\− mice appeared to less than that of WT mice, though the difference did not reach statistical significance.

We next sought to investigate the CYP KO effects in CS-exposed mice. Whole-body CS exposure was performed. Briefly, mice were exposed to CS for 4 hours per day for 6 weeks. Exposure was accomplished using a smoking chamber (Teague Enterprises, Davis, CA). We determined the LD₅₀ of IAV PR8 in WT mice (1000 PFU/mouse) and all mice were inoculated with this dose (FIGS. 2C and 2D). CS exposure increased morbidity and mortality of IAV infection in mice, with CS-exposed mice having a much lower survival rate than NS mice after IAV infection (FIG. 2C; 0% for WT CS vs. 40% for WT NS mice). However, CYP KO mice exposed to CS had significantly improved survival compared to CS-exposed WT mice during IAV infection (40% survival for CYP1B1 KO CS vs. 0% for WT CS, p<0.05, log rank test). The survival rate of CS-exposed CYP KO mice was recovered to that seen in WT NS mice. Morbidity was also decreased, as CYP KO mice had significantly less weight loss compared to WT mice at day 8 and 9 post-infection (FIG. 2D), with weight loss being restored back to that seen in WT NS IAV-infected mice. Thus, the data demonstrated that CYP KO significantly improved morbidity and mortality in CS-exposed mice during IAV infection.

### CYP KO Increased Immune Cell Influx in BALF and Less Lung Injury in NS Mice During IAV Infection

We then sought to determine whether CYP KO decreased lung injury during IAV infection. Mice were inoculated intranasally with IAV at 500 PFU/mouse. The mock group was sham inoculated with an equal volume of PBS as a negative control. Animals were sacrificed by overdose isofluorane at 5 days p.i. We first determined the total inflammatory cell numbers in bronchoalveolar lavage fluids (BALF). IAV infection increased total viable leukocyte number in BALF in WT NS mice. This increase was further enhanced in CYP KO mice infected with IAV whether or not they were exposed to CS.

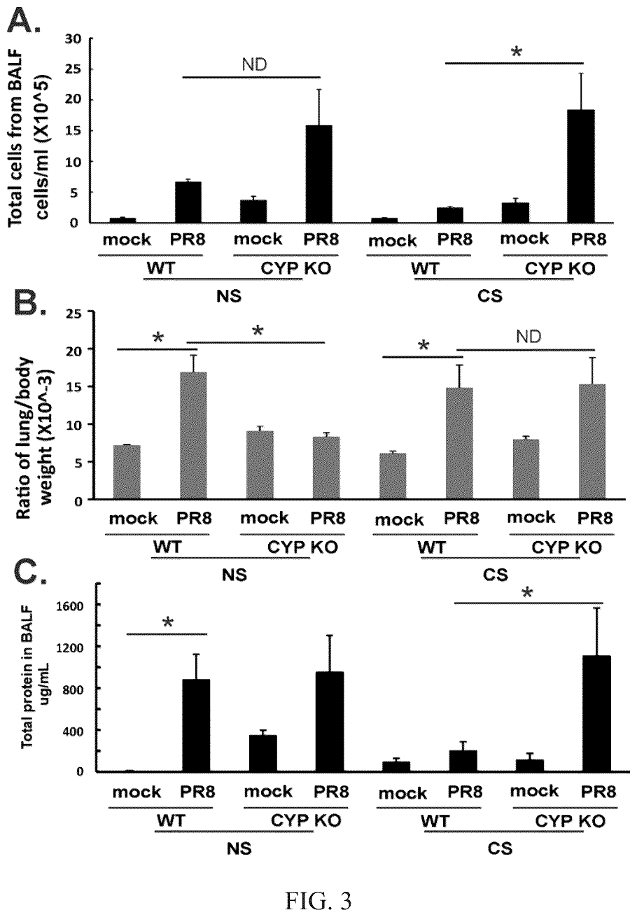

The increase in BALF leukocyte number happened in CS-exposed CYP KO mice (FIG. 3A) even though CS decreased total BALF cell numbers during IAV infection in these experiments. These results showed that CYP KO increased immune cell influx into the lung in both NS and CS-exposed mice during IAV infection. The total amount of protein in the BALF was also increased in all CYP KO groups relative to similarly treated WT mice (FIG. 3C). These data indicated that an impairment of recruitment mediated by CS was likely related to higher mortality of CS-exposed IAV-infected WT mice. However, CYP KO mice recruited more inflammatory cells to the lung, potentially correcting innate immune response impairment caused by CS.

The lung-to-body weight ratio (LBR) is relevant in assessing lung health and function, serving as an indicator for lung injury. In WT mice, IAV infection significantly increased LBR in both NS and CS mice (FIG. 3B). However, CYP KO significantly decreased this ratio to that seen in mock-infected WT mice. In CS mice, LBR in CYP KO and WT mice were similar after infection (FIG. 3B). Thus, CYP KO increased cellular influx and decreased lung injury in IAV-infected NS mice compared to levels seen in uninfected mice. In CS-exposed mice, KO increased cellular influx and did not cause more lung injury than WT mice.

### Effect of CYP KO on Lung Histopathology During IAV Infection

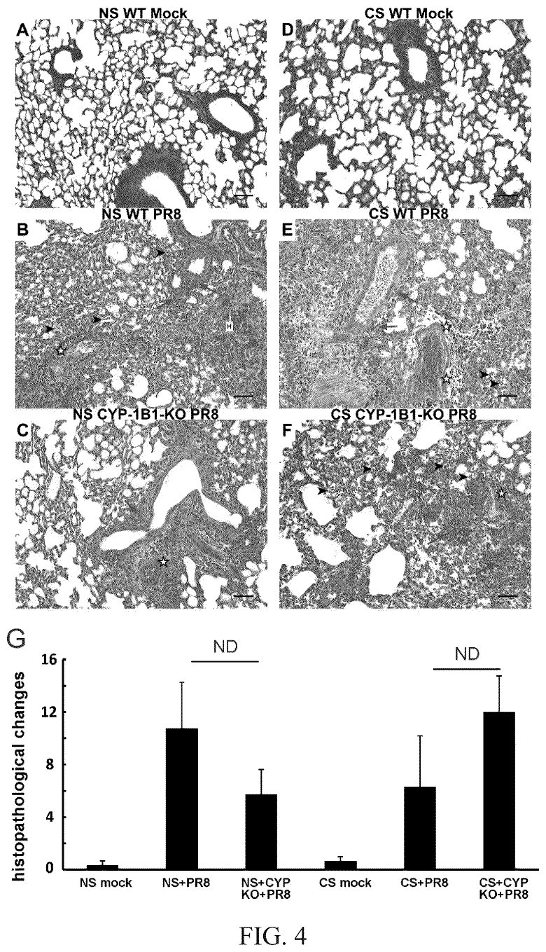

Lung tissues were evaluated for histopathological changes. At day 5 p.i., IAV-infected mice displayed a typical histopathological pattern for viral pneumonia, including diffuse alveolar damage (DAD) such as marked alveolar edema, fibrin exudation, hemorrhage, and prominent hyaline membrane formation. There were severe inflammation of bronchi and bronchioles characterized by epithelial cell necrosis and sloughing with partial to complete airway obstruction by intact and degenerate neutrophils and cellular debris, and varying degrees of acute intra-alveolar edema and/or hemorrhage. Lung histopathology of mock infected control mice was unremarkable and within normal limits. Histopathologic scoring of these cardinal features of IAV infection was evaluated by a pathologis blinded to the treatment groups. Mostly consistent with the lung-to-body ratio data, NS CYP KO+PR8 mice had lower overall pathologic scores than the NS WT+PR8 group (FIG. 4G), though this difference did not reach statistical significance. In contrast, CS CYP KO+PR8 mice had increased histopathologic scores compared to CS WT+PR8 mice, though again this difference did not reach statistical significance. The results suggest that, though CYP KO altered immune cell influx and lung injury as determined by cell counts, BALF protein, LBR and histopathologic score, these alterations by themselves do not explain the differences in mortality seen in CYP KO mice.

### CYP1B1 KO Induced Early IFN-γ Production in the Lung During IAV Infection

To examine additional mechanisms whereby CYP KO improved outcomes in IAV-infected mice, we measured expression of the pattern recognition receptors that recognize viruses and their downstream interferon expression during IAV infection in all mouse groups. Mice were inoculated intranasally with a single dose of the IAV PR8 strain (500 PFU). Lung tissues and BALF were collected at 5 days after infection. mRNA or protein expression was determined by qRT-PCR or multiplex immunoassay, respectively.

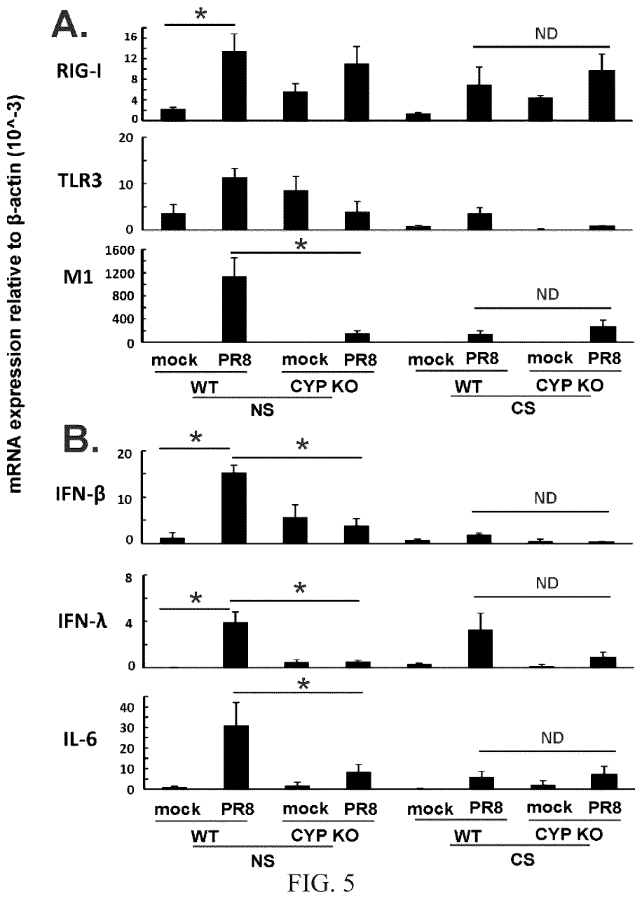

CS-exposed WT mice had suppressed RIG-I and TLR3 mRNA induction by IAV compared NS WT mice (FIG. 5A; NS WT PR8 vs. CS WT PR8). CYP KO mice had similar RIG-I mRNA induction by virus compared to WT mice in both NS and CS mice (FIG. 5A). For NS mice, CYP KO mice had significantly less IFN-β and IFN-λ than those in WT mice (FIG. 5B), which might explain the decrease in lung injury seen in these NS CYP KO PR8 mice (LBR, FIG. 3B). NS WT PR8 mice had the most robust induction of the proinflammatory cytokine IL-6 mRNA induction among all the groups (FIG. 5B). NS CYP KO PR8 mice significantly expressed less IL-6 compared to the NS WT PR8 group, and again this may have contributed to the decrease in lung injury seen in NS CYP KO PR8 mice (LBR, FIG. 3B). In terms of viral RNA expression, the NS WT PR8 mouse group had the most IAV M1 protein mRNA expression of any treatment group, while the NS CYP KO PR8 group had significantly less viral replication, which indicated CYP KO might be helpful in controlling viral replication in the lung, at least in NS mice (FIG. 5A).

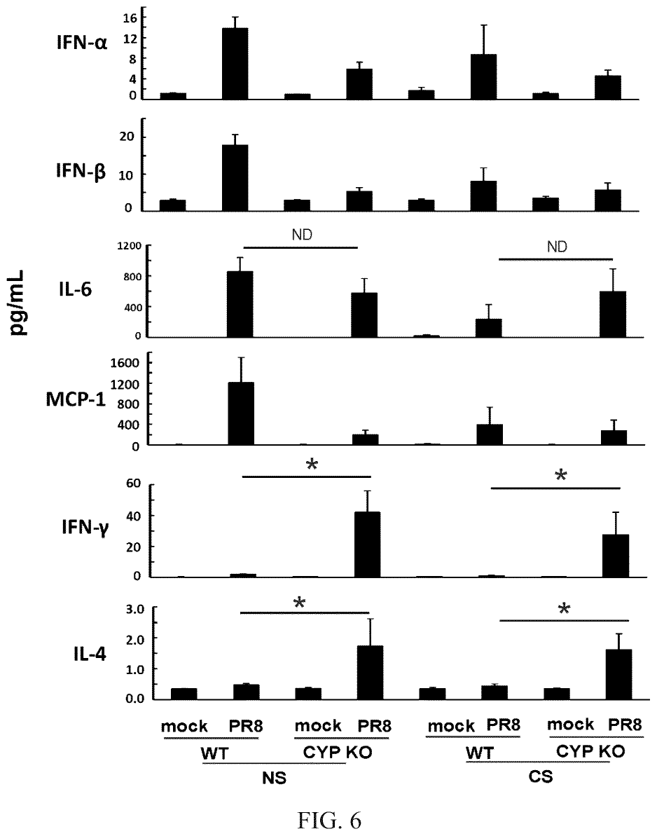

Since both IFN-β and IFN-λ mRNA expression levels were low, we next sought to find out the cause of the immune cell influx at day 5 p.i. as we showed in FIG. 3A. We measured pro-inflammatory cytokine protein levels in mouse BALF in all groups using multiplex immunoassay (FIG. 6). Consistent with the mRNA expression data, IFN-α and IFN-β protein induction by IAV were lower in CYP KO mice relative to WT cohorts in both NS and CS mice (FIG. 6). Cytokine IL-6 protein levels were similar among all infected groups. Cytokine MCP-1 protein level induction by IAV was also reduced in CYP KO mice. Remarkably, IFN-γ levels were significantly increased in CYP KO mice relative to those seen in WT mice. Interestingly, the anti-inflammatory cytokine IL-4 protein levels were also significantly induced in CYP KO regardless of NS or CS (FIG. 6). Thus, these results demonstrate that CYP KO in mice boosted the pro-inflammatory cytokine IFN-γ response and anti-inflammatory cytokine IL-4 induction at day 5 after IAV infection.

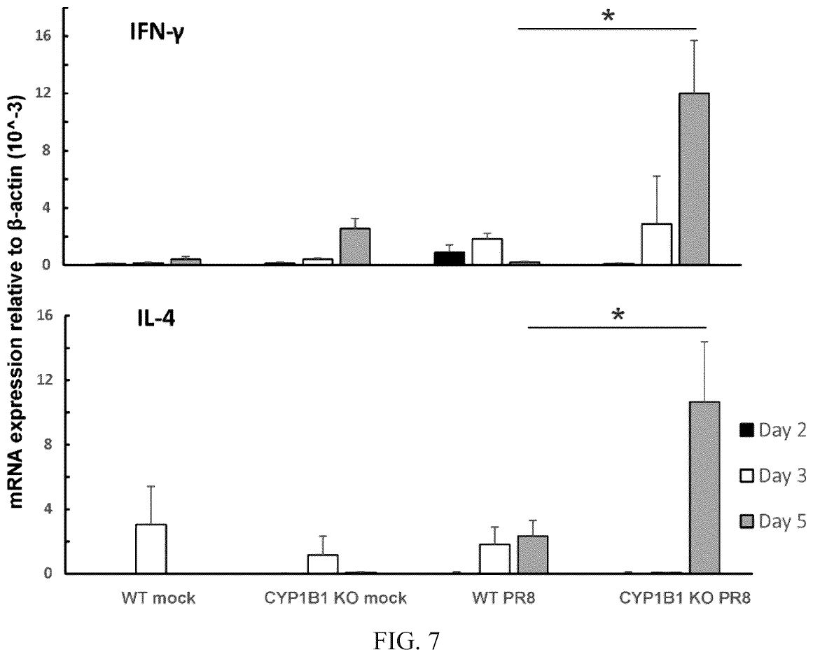

To determine the early kinetics of IFN-γ induction in CYP KO mice, we infected WT and KO mice and collected lungs at day 2, 3 and 5 after infection. We found that IFN-γ was similarly induced in IAV-infected WT and KO mice at day 3 p.i. (FIG. 7). At day 5 p.i., CYP KO produced significantly greater IFN-γ mRNA in response to infection than IAV infected WT mice did. IL-4 mRNA induction was also significantly enhanced by viral infection in KO mice at day 5.

CYP KO Mice Recruited More IFN-γ-Producing NK Cells and Myeloid Cells in the Lung after IAV Infection

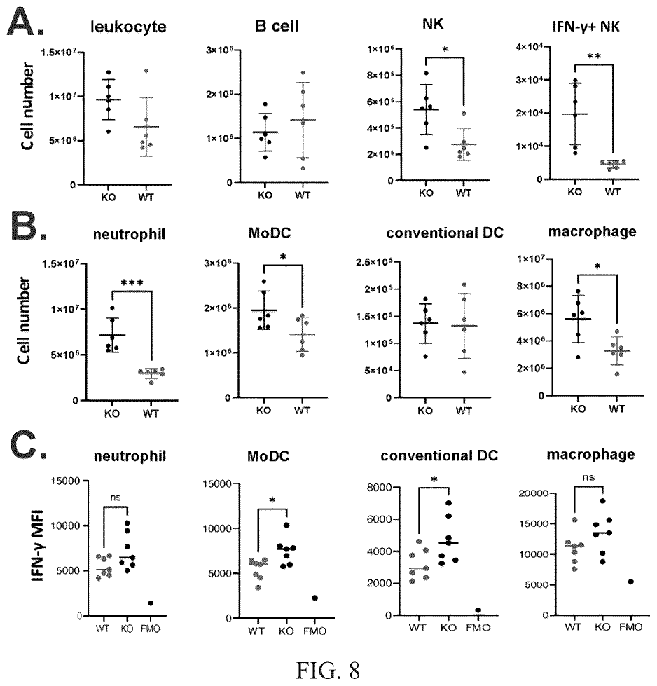

To determine which cell(s) contribute to the increased IFN-γ, total lung cells were isolated at 5 days after IAV infection. We measured lymphoid cell and myeloid cell numbers in WT and KO mice by flow cytometry. We found total leukocyte numbers were higher in KO mice as we found in BALF in FIG. 3A (FIG. 8A). Lung NK cells are usually considered as the major source of IFN-γ in early stage of IAV infection. We indeed found that NK and IFN-γ+NK cell numbers were significantly higher in KO mice than in WT cohorts. Surprisingly, CYO KO mice also had significantly higher numbers of myeloid cells including neutrophils, MoDC and macrophages during IAV infection (FIG. 8B). To further examine the source of the increase per cell in the production of IFN-γ, we also measured Mean Fluorescence Intensity (MFI) of IFN-γ in these myeloid cells. MoDC and conventional DC both had significantly increased IFN-γ MFI in KO mice than those in WT mice (FIG. 8C). The data suggest that, besides classical innate IFN-γ producer NK cells, myeloid cells are involved in the enhanced production of IFN-γ in CYP KO mice after IAV infection.

### IFN-γ is Required but not Sufficient for Protection of CS-Exposed Mice Against Lethal Influenza Virus Infection

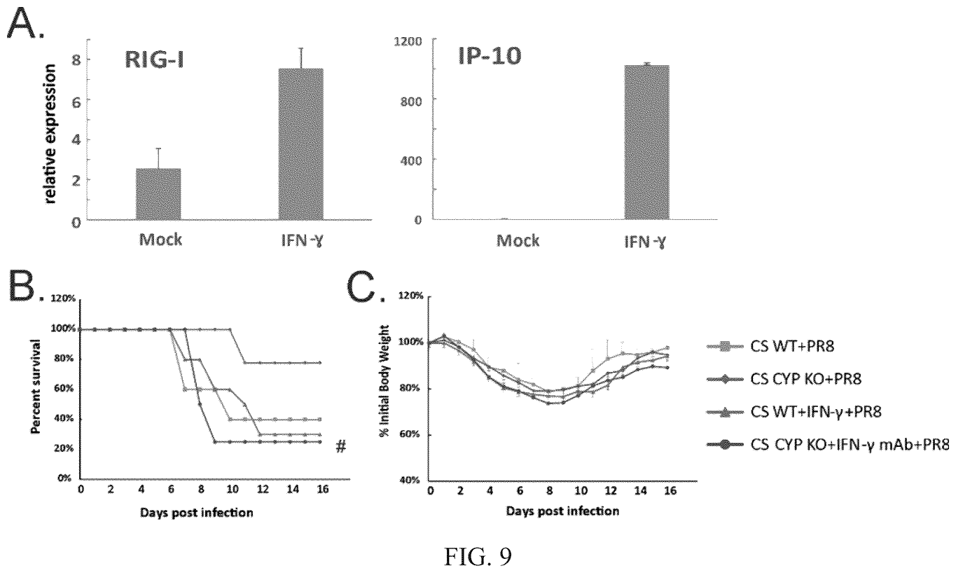

To determine whether IFN-γ induction may play an important role in the protection of the CS-exposed animals from IAV infection in KO mice, we next examined whether exogenous IFN-γ administration improved outcomes in CS-exposed IAV-infected WT mice. First, we confirmed that recombinant mouse IFN-γ (Biolegend, San Diego, CA) administration induced innate immune responses in non-infected animals. Mice were lightly anesthetized with isoflurane and treated with IFN-γ (5 μg/mouse) intratracheally in a total volume of 50 μl in PBS. Mock groups were sham treated with a single dose of intranasal sterile PBS solution (diluent). We found that IFN-γ induced robust innate immune responses in the mouse lung, including increased mRNA of RIG-I. Significantly, mRNA levels of interferon gamma-induced protein 10 (IP-10, a cytokine downstream of IFN-γ) were induced 426-fold over mock by IFN-γ administration (FIG. 9A).

We next evaluated the effect of IFN-γ administration on mortality and weight loss in CS-exposed, IAV-infected WT mice. We experimentally determined an LD50 for CS-exposed mice and discovered this dose was 500 PFU/animal. We used this dose in CS-exposed mice with or without IFN-γ administration (FIGS. 9B and 9C). IFN-γ was given to mice intratracheally at day 3 after IAV infection. We found IFN-γ treated CS WT mice had almost the same mortality as untreated CS WT IAV-infected mice (FIG. 9B), which suggested IFN-γ administration alone did not protect the CS-exposed WT mice from lethal IAV infection. We also tested whether blocking IFN-γ abolishes the protection in CYP KO mice during IAV infection. Mice were intraperitoneally injected with 600 μg anti-IFN-γ monoclonal antibody (mAb) on days 2, 4, 6 p.i. IFN-γ neutralization significantly decreased survival at the LD₅₀ for CS-exposed mice (80% for CS CYP KO+PR8 vs. 20% for CS CYP KO+IFN-γ Ab+PR8 mice). The data suggested that IFN-γ was required, but not sufficient for protection of CYP KO CS mice during lethal IAV infection.

Examples of CYP1B1 inhibitors that may be used in the methods of the present disclosure include but are not limited to those shown in Tables 2 and 3.

TABLE 2
Examples of CYP1B1 inhibitors

1,2-,1,3- and 1,4-phenylenebis(methylene)selenocyanate
α-naphthoflavone, acetylenes, 2-ethynylpyrene,
hesperetin, homoeriodictyol, acacetin, diosmetin
resveratrol
oltipraz
2,4,3′,5′-tetramethoxystilbene
hydroxystilbenes
flutamide, pacilitaxel, mitoxantrone, docetaxel, tamoxifen, doxorubicin,
daunomycin
2,3′,4,5′-Tetramethoxystilbene, trans-Stilbene analogues
imperatorin, isopimpinellin
purpurin, alizarin
polycyclic aromatic hydrocarbons
pyricetin, apigenin, kaempferol, quercetin, amentoflavone, quercitrin,
rutin
trans-resveratrol methyl ethers
3′,4′-dimethoxyflavone, 5,7,4′-trimethoxyflavone, curcumin7,4′-
dimethoxyflavone, 7,3′-dimethoxyflavone, quercetin
thiomethylstilbenes
2,2′,4,6′-Tetramethoxystilbene
methoxyflavonoids
melatonin
2,3,4-trimethoxy-4′-methylthio-trans-stilbene
propargyloxyflavones
2-(4-(3′-fluoro-6,7,10-trimethoxy-α-naphthoflavonol)octyloxy) -
2-oxoethanaminium chloride

TABLE 3
Examples of CYP1B1 inhibitors, con't.

ticagrelor
bosutinib
brinzolamide
ceforanide
xipamide
dasatinib
ezetimibe
ozenoxacin
acotiamide-d₆
trichlormethiazide
vilazodone
darolutamide
donepezil
haloperidol
dolutegravir
cabergoline
isavuconazole
ceftezole
pantethine
lapatinib
pyritinol
(−)-epigallocatechin
gallate
D-α-tocopheryl acetate
pexidartinib
lenvatinib
ozanimod
halofuginone
nebivolol
gefarnate
doripenem
glyceryl trioctanoate octanoic acid
peimine
glibenclamide
chlorprothixene
nadifloxacin

### DISCUSSION

The present disclosure demonstrates that CYP1B1 is one of the most highly upregulated (6-fold over nonsmokers) genes in lung epithelial cells obtained from smokers (Table 1). With regards to IAV infection, we found that CYP1B1 mRNA expression is induced during IAV infection, and is further enhanced by CS exposure. These results suggest a correlation between increased CYP1B1 expression and increased mortality during IAV infection with CS exposure.

We discovered that CYP KO significantly increased survival during IAV infection in CS-exposed mice. Further investigation revealed that CYP KO significantly increased IAV-induced total immune cell numbers in BALF without causing additional lung injury. Notably, we found that CYP KO significantly increased early IFN-γ induction in the lungs at day 5 p.i. in both NS and CS-exposed mice. IFN-γ, also known as type II IFN, is a potent antiviral cytokine that plays multiple roles in both innate and adaptive immune responses to IAV infection. Mice lacking IFN-γ or its receptor are highly susceptible to infectious diseases, emphasizing its importance. IFN-γ stimulates the recruitment of both innate and adaptive leukocytes to the infection site. Enhanced induction of IFN-γ in CYP1B1 KO mice can explain the reason that total immune cell numbers in BALF were elevated in these mice.

There are two consequences of an imbalanced immune response to viruses, insufficient antiviral activity or excessive inflammation and tissue damage. As we have shown previously there is suppressed immune cell recruitment in the lungs of CS exposed, compared to NS mice during IAV infection. Thus, CS impairs cellular antiviral activity at the early stage of IAV infection. Enhanced recruitment of immune cells to the lung of CYP KO mice appears to partially reverse the immune defect in CS-exposed mice, but may not be beneficial in NS mice where recruitment is normal.

Additionally, IFN-γ signaling increases the presentation of antigen to CD8 and CD4 T cells via the major histocompatibility class I (MHCI) and MHCII pathways, respectively. This promotes T cell activation and ultimately aids in virus clearance. We have shown previously that CD8 and CD4 T recruitment to the lungs is suppressed at the early stages of IAV infection in CS-exposed mice. Early IFN-γ induction in CYP KO mice might help the host recruit sufficient CD8 and CD4 T cells to the lungs to enhance viral clearance and improve outcomes.

For WT mice, innate lymphoid cells, particularly NK cells, were the dominant producers in IFN-γ induction, both in terms of numbers of cells and amount of cytokine produced, at the early stage after IAV infection. In IFN-γ−/−mice, DCs and T cells show decreased migration to the lymph nodes and limited influenza-specific responses in the lung. This is rescued by adoptive transfer of WT NK cells; NK cell-derived IFN-γ alone is sufficient for T cell activation during influenza infection, while T cell-derived IFN-γ is complementary. The present data confirmed that NK cells are a major producer of early IFN-γ release in CYP KO mice (FIG. 8). Additionally, we found that, besides NK cells, myeloid cells including neutrophils, MoDC, conventional DC and macrophages might also contribute to early IFN-γ production in the IAV-infected CYP KO mice.

We confirmed the importance of IFN-γ for CYP KO survival by adding IFN-γ antibody to CYP KO mice. IFN-γ antibody treatment of CYP1B1 KO mice completely abolished the higher survival rate in the CS-exposed IAV-infected mice. Additionally, IFN-γ administration to CS-exposed WT mice did not increase survival rates as seen in CYP KO mice. The data suggested that early IFN-γ is one of the multiple factors involved in improved outcomes in KO mice. Thus, for CS-exposed CYP1B1 KO mice, IFN-γ is necessary but insufficient for enhanced protection against fatal IAV infection.

The present results indicate that CYP1B1 is one of the most highly upregulated genes in human and mouse lungs exposed to CS. CYP1B1 KO in mice is protective for CS enhanced susceptibility of smokers during influenza infection. IFN-γ is important in the mechanism of protection. IFN-γ is required but is not sufficient for the protection of CS-exposed CYP KO mice against lethal influenza virus infection.

What is claimed is:

1. A method of treating an influenza infection in a subject in need of such treatment, comprising administering a CYP enzyme inhibitor to the subject.

2. The method of claim 1, wherein the influenza infection is caused by an influenza A virus, an influenza B virus, or a combination thereof.

3. The method of claim 2, wherein the influenza A virus is selected from H1N1, H3N2, or an avian influenza strain.

4. The method of claim 1, wherein the CYP enzyme is CYP1A1 or CYP1B1.

5. The method of claim 1, wherein the CYP enzyme is CYP1B1.

6. The method of claim 1, wherein the subject has increased expression of a CYP1B1 gene product.

7. The method of claim 1, wherein the subject is a tobacco smoker or is a secondhand smoker.

8. The method of claim 1, wherein the tobacco smoker is a cigarette smoker.

9. The method of claim 1, wherein the subject exhibits one or more symptoms selected from fever, cough, sore throat, myalgia, fatigue, nasal congestion, or dyspnea.

10. The method of claim 1, wherein the CYP enzyme inhibitor increases immune cell infiltration into the lung.

11. The method of claim 10, wherein the immune cells are NK cells and/or IFN-γ+NK cells.

12. The method of claim 10, wherein the CYP enzyme inhibitor increases type II IFN production.

13. The method of claim 12, wherein the type II IFN is IFN-γ.

14. The method of claim 1, wherein the CYP enzyme inhibitor is administered orally, intravenously, intranasally, intramuscularly, or subcutaneously.

15. The method of claim 1, wherein the CYP enzyme inhibitor is administered once daily, twice daily, or in a sustained-release formulation.

16. The method of claim 1, wherein the CYP enzyme inhibitor is administered at a dose between 0.1 mg/kg and 50 mg/kg.

17. The method of claim 1, wherein the CYP enzyme inhibitor is formulated with a pharmaceutically acceptable carrier.

18. The method of claim 1, further comprising administering an antiviral agent selected from oseltamivir, baloxavir, zanamivir, or peramivir.

19. The method of claim 18, wherein the CYP enzyme inhibitor and the antiviral agent are co-administered or sequentially administered.

20. A pharmaceutical composition comprising a CYP enzyme inhibitor; an antiviral agent selected from oseltamivir, baloxavir, zanamivir, or peramivir; and a pharmaceutically acceptable carrier.
